(19) United States
(12) Patent Application Publication
(10) Pub. No.: US 2017/0209627 A1
(43) Pub. Date: Jul. 27, 2017

(54) Medical Device With Enhanced Visibility

(71) Applicant: Plass Medtech AG, Stetten (CH)

(72) Inventor: André Plass, Zurich (CH)

(21) Appl. No.: 15/327,985

(22) PCT Filed: Jul. 16, 2015
(86) PCT No.: PCT/EP2015/066320

§ 371 Date: Jan. 20, 2017

(30) Foreign Application Priority Data
Jul. 23, 2014 (CH) 1141/14
Jun. 19, 2015 (CH) 881/15

(51) Int. Cl. A61L 31/10; B05D 3/06; B05D 1/18; B05D 1/28; A61L 31/14; B05D 1/02

(52) U.S. Cl. CPC A61L 31/10; A61L 31/14; B05D 1/02; B05D 1/18; B05D 1/28; B05D 3/067; A61L 2300/442; A61L 2420/02; A61L 2420/08

(57) ABSTRACT

A medical device (1) with enhanced visibility for insertion into a human or animal body and/or for contact with human or animal tissue is provided, comprising a main body (10) and at least one first coating (11); said at least one first coating (11) at least partially covering the main body (10); and said at least one first coating (11) comprising an at least partially translucent matrix (111) and at least one luminescent component (112).

### FIELD OF THE INVENTION

The present invention relates to medical devices with enhanced visibility, in particular for contact with tissue and/or for insertion into a human or animal body.

### BACKGROUND OF THE INVENTION

In hospital environments, the ability of efficient and accurate orientation and navigation for the operator is an important requirement for successful medical treatments and interventions. However, this can often be impeded by several factors. In operating rooms for example, the general setting such as a plurality of medical equipments and tools standing in the room and obstructing lighting or illumination can make efficient navigation difficult. Other examples of impeding factors could be hospital environments in developing countries or power failures.

On the other hand, treatments or interventions can occur outside of a hospital environment, for example in an emergency situation, where the setting is usually not controllable and can be unfavourable for medical treatments or interventions. In addition, sufficient assistance may not be available in such an emergency situation, which makes the treatment for a single operator without assistance difficult.

### SUMMARY OF THE INVENTION

With respect to orientation and navigation, appropriate lighting conditions are an important factor for successful medical treatments and interventions. However, differing demands in the specific medical environment can result in lighting conditions which are not ideal for each step of a medical treatment. In operating rooms or catheterization labs the lighting can be intentionally dimmed in order to enable a better view to the screens during the interventions. As a result, however, the visibility of medical devices used is decreased which can in turn complicate the interventions.

This is also the case for medical treatments or interventions occurring outside of a hospital environment where the lighting conditions are not controllable and can be unfavourable.

An example of an intervention technique where appropriate lighting conditions can play an important role is the Seldinger technique which is an important platform technique for almost all percutaneous interventions. Unfavourable lighting conditions, for example in an operating room with dimmed lighting, can hinder the visibility of the wire used in said technique and make an efficient navigation by the operator difficult.

It is therefore an object of the invention to provide a medical device which enables improved navigation and orientation by a user of said medical device.

This object is achieved by the subject matter of the independent claims. Exemplary and/or favourable embodiments are further defined by the dependent claims and the disclosure of this document.

According to an aspect of the invention, the object is achieved by a medical device with enhanced visibility for insertion into a human or animal body and/or for contact with human or animal tissue. The medical device comprises a main body and at least one first coating. The at least one first coating at least partially covers the main body. In general, said at least one first coating comprises an at least partially translucent matrix and at least one luminescent component.

In the context of this document, a material is understood to be “luminescent” if it emits visible radiation by the transition from an excited quantum state to a ground state. The energy necessary for the transition from the ground state to the excited state is preferably provided by electromagnetic radiation, preferably visible light. Exemplary forms of luminescence are phosphorescence or fluorescence.

The at least one luminescent component of the at least one first coating contributes to the effect that the medical device exhibits an enhanced visibility even in cases where the lighting conditions are not optimal. Said medical device is therefore particularly suited for use in operating rooms with dimmed lighting or in an outdoor environment, for example in emergency situations.

The at least one luminescent component may be embedded in the at least partially translucent matrix. The at least partially translucent matrix ensures that the light which is emitted by the luminescent component is not or only weakly damped.

The fact that the at least one luminescent component is comprised in a coating has the advantage that medical devices according to the present invention may keep the geometrical shape of conventional medical devices which are each already optimized for its purpose. Conventional medical devices may be equipped with the first coating according to the present invention in order to obtain a medical device with enhanced visibility.

In general, the at least one first coating may comprise several different luminescent components. A suitable combination of different luminescent components may enhance the visibility and/or the contrast and may enable to achieve different colours of the at least one first coating.

In a preferred embodiment, the medical device further comprises at least one second coating, said second coating comprising an at least partially translucent matrix. In general, said second coating encases the at least one first coating.

The at least one second coating may serve as a protection coating encasing the at least one first coating. By encasing the at least one first coating, the at least one second coating also encases those parts of the main body of the medical device which carry the at least one first coating. For example, the tip of a needle may be coated by a first coating comprising a luminescent component. Said first coating may be encased by a second coating which serves as a protection coating preventing the first coating, e.g. from wear. The aspect of the protection of the at least one first coating by the at least one second coating may be particularly important for the medical use since the first coating may comprise components which are not biocompatible. In order to ensure sufficient protection, the at least one second coating is preferably continuous. Preferably, the second coating may not exhibit any cracks, or holes or bubbles. Furthermore, the second coating has preferably a uniform thickness and firmly bonds with the at least one first coating. In order to transmit most of or all light emitted from the at least one luminescent component of the at least one first coating, the at least one second coating preferably comprises an at least partially translucent matrix.

In an embodiment, the second coating comprises at least one luminescent component.

The at least one luminescent component of the second coating may be different from the luminescent component of the at least one first coating. A combination of different luminescent components within the at least one first and/or second coating or between the at least one first and/or second coating may enable enhanced visibility or may enable different colour appearances of the medical device.

In a preferred embodiment, the matrix of the first and/or second coating is biocompatible. The matrix preferably comprises one or a combination of the following materials: Ethylene tetrafluoroethylene (ETFE), Polytetrafluoroethylene (PTFE), Polyesterimide, Polyimide.

In order to be used in medical treatments and/or interventions, the medical device should be biocompatible. This may be achieved by the first and/or the second coating being biocompatible. In particular, the medical device according to the present invention may fulfil the biocompatibility test matrix according to ISO 10993-1.

Preferably, the at least one luminescent component comprises fluorescent and/or phosphorescent particles. Such particles may be for example one of the Storelite® products obtained by RC Tritec AG, Switzerland, such as particles from the Storelite HS series or Storelite RNS pigments. A combination of different fluorescent and/or phosphorescent particles may be used within a coating in order to optimize the luminosity and/or the level of contrast. For example, the addition of fluorescent yellow pigments may decrease the luminosity but increase the visibility at daylight. The afterglow characteristics may be optimized to achieve the maximal luminescence during the first hour after exposure to light. The use of phosphorescent particles for the coating has the advantage that a self-glowing medical device may be provided which glows and ensures an enhanced visibility for a defined period of time after light exposure. The addition of fluorescent particles may significantly increase the visibility while UV lighting and also under regular light conditions.

The fluorescent and/or phosphorescent particles may be of a size of between 10 microns (μm) and 30 microns (μm). The single particles are sized for optimal luminosity and optimal suitability for encasement by a second coating.

In an embodiment, the matrix of the first and/or second coating comprises enamel varnish, preferably PTFE or ETFE. The enamel varnish may have the advantage that established enamelling techniques can be used for the present invention. PTFE or ETFE may be advantageous to ensure biocompatibility.

An enamel varnish composition may be used to form the enamel varnish.

The enamel varnish composition may be mixed with the luminescent component. Preferably, the weight ratio of the luminescent component to the enamel varnish is in a range of 0.1:1 to 2:1. Preferably, the mixing ratio is 1:1. The advantage of mixing the luminescent component with the enamel varnish composition is that a more regular coating may be achieved. The ratio of the mixture and/or the size of the fluorescent and/or phosphorescent particles may be varied to achieve a different smoothness of the coating and/or to achieve different luminosities.

According to an embodiment, the matrix of a coating composition for the first and/or second coating comprises a thinner, preferably Xylene. The thinner may be used to vary the viscosity of the coating composition. This can be advantageous to adapt the coating composition to the different coating processes disclosed in this document. The thinner may be 2 to 15 weight % of the mixture of the enamel varnish composition and the luminescent component. In some embodiments, the thinner may vary between 2 to 15 of weight % of the mixture depending on the viscosity of the coating composition.

The main body of the medical device may be a wire or a needle or a tube or a rod. Preferably, the main body is made of metal.

In other embodiments, the main body of the medical device may be a suture, a probe, an electrode, a clamp or any other surgical instrument.

The advantage of a main body being made of metal is that a coating or coating composition may exhibit improved adhesion on the surface of the surface compared to other materials. In general, however, the coating composition as disclosed in this document may be applicable and/or adaptable to any other material used for the production of medical devices.

An advantage of the present invention is that a coating composition comprising a luminescent component as disclosed in this document may in principle be applied to any material and main body of the medical device. The present invention therefore provides a very versatile solution to enhance the visibility of such medical devices.

In an embodiment, the medical device is flexible. In this context, flexibility may be understood as the ability of the medical device to be bendable or elastically deformable.

This may be the case for embodiments where the main body of the medical device is a wire, such as for example a wire used in the Seldinger technique.

In order to allow the medical device to substantially maintain the flexibility of the main body, the coating of the medical device may be chosen such that the flexibility of the medical device is sustained. The flexibility of the coating may substantially be influenced by the properties of the matrix, which is flexible to a sufficient degree. In particular, the medical device according to the present invention may be bendable without breaking or cracking the coating. It is furthermore known to the person skilled in the art that the flexibility of the coating is not significantly changed by the presence of the luminescent components, such as fluorescent and/or phosphorescent particles.

According to an embodiment, the medical device further comprises visible marks indicating spatial distances on the medical device.

Visible marks may be advantageous in embodiments where the main body of the medical device is a wire used in the Seldinger technique. In those embodiments, the visible marks may serve as length indications. Thereby by simply looking at the marks, the operator gets immediately the information on the length of the part of wire which is inserted into the body.

The visible marks may exhibit different colours. Preferably, the colours may yield a certain colour coding known to the operator and providing additional information.

The visible marks may comprise luminescent components such that the marks may be visible also under poor lighting conditions.

According to a further aspect of the present invention, the object is achieved by a method of coating a medical device as disclosed in this document, characterized by the steps: (a) Providing a main body, (b) providing a coating composition by mixing a matrix material and at least one luminescent component, (c) applying the coating composition to at least a part of the main body, (d) finishing the coating composition.

In some preferred embodiments, the coating is generated by paint enamelling and/or dipcoating and/or spraycoating and/or brushcoating and/or powdercoating.

The finishing of the coating composition may be achieved by curing with UV light and/or blue light and/or air and/or heat, for example in a furnace.

In an embodiment, the method of coating the medical device may further be characterized by a step of generating visible marks at specific positions on the medical device, preferably by laser treatment and/or by painting and/or spraying and/or burning. The visible marks may indicate spatial distances at specific positions on the medical device.

In some embodiments, a further coating composition is provided and applied to the coated medical device. In this way, the medical device can comprise several layers of coating which may allow to vary the flexibility and the luminescent properties.

In the following, some examples of methods of coating a medical device according to the present invention will be given. The examples refer to an embodiment where the main body of the medical device is a wire. It is clear to the person skilled in the art that any other main bodies, for example those disclosed in this document, are suited for the methods given by the following examples.

### A) Paint Enamelling

Preparation of the wire: The wire is cleaned with an ultrasonic device using at least three cleaning media. The first cleaning step is carried out with a degreasing agent like white spirit or the like. The second cleaning medium is a water-soluble liquid soap diluted with 80-93% of water and the third is pure ethanol.

Coating process: The coating composition is applied in several layers to ensure a uniform coating avoiding pinholes or other irregularities. First the clean wire is passed through a textile felt, which is soaked with white PTFE which serves as an enamel composition. After passing the felt a thin layer of PTFE will remain on the wire surface. The layer has an approximate thickness of 5 μm. Immediately after the application of the PTFE the wire will be passed into an on-line furnace to cure the enamel composition (PTFE). The curing temperature can vary from 300° C. to 500° C., depending on the type and fluidity of the coating composition and the processing speed. Both, speed and temperature must be set accordingly to obtain a correct polymerization. In the present example, the wire passes with a speed of 2-3 meters per second through the felt and the furnace. Then approximately 4-6 layers of an enamel mixture of clear PTFE with fluorescent and/or phosphorescent particles mixed at a ratio of 1:1 are applied to the wire to achieve a first coating. A thickness increase of the coating of 40-70 μm in total is thereby achieved. To adjust the viscosity of the enamel composition a thinner is used. Xylene is applied until the necessary fluidity for the process is achieved. In the present example, the thinner is around 2% of the final weight of the 1:1 ratio mixture. The wire will pass the furnace with the same speed and temperature as before. As soon as the desired diameter is achieved and the surface of the coating has an even and regular appearance, additional two layers of clear PTFE without the fluorescent and/or phosphorescent particles are applied in the same manner to achieve a second coating to protect the particles and to obtain a biocompatible surface.

Quality control: To check if the coating does not peal off the wire it is stretched to the breakage point with a tensile strength measuring machine. If the coating peals from the wire at the breakage point and leaves behind a hollow tube of coating or flakes, the coating is insufficient. In this case the curing process in the furnace must be adjusted varying speed and temperature to obtain a good result.

### B) Spray Coating

The cleaning, coating and quality control processes are the same as for the paint enamelling example. However, the enamel composition is applied with a spray nozzle or an ultrasonic nebulizer to the wire and not with a felt. To achieve a viscosity which is suitable for spray coating, the amount of thinner is around 15% of the weight of the enamel composition. Therefore thicker layers can be applied to the wire, approximately 5 to 10 μm per layer. The wire is again passed through a furnace, but at a lower speed of 1-2 meters per second. The temperature of the furnace remains at 300-500° C. If spraying is used, only 1-2 layers of clear PTFE with fluorescent and/or phosphorescent particles are applied as a first coating, because the final diameter desired is achieved faster. The final layers are again clear PTFE without fluorescent and/or phosphorescent particles as a second coating.

### C) Dip Coating

In this example, the wire is dipped in the enamel composition and with a regular motion the wire is pulled out of the enamel composition. To achieve a viscosity which is suitable for dip coating, the amount of thinner applied is around 3% of the weight of the enamel composition. The layers are achieved faster as with the paint enamelling example. The same amount of layers and the same thicknesses of layers can be achieved as with spray coating. The same speed and the same temperature as for the spray coating process is applied to cure the enamel PTFE on the wire.

### LIST OF FIGURES

Embodiments of the invention will be better understood from the detailed description given herein below and the accompanying drawings. The drawings are showing:

FIG. 1 a perspective view of an embodiment of the medical device with a wire as a main body;

FIG. 2a a cut view of the medical device along line A-A in FIG. 1;

FIG. 2b a magnification view of the circle marked by C in FIG. 2a; and

FIG. 3 a cut view of the medical device along line B-B in FIG. 1.

### DETAILED DESCRIPTION OF EXEMPLARY EMBODIMENTS

For the purposes of illustrating the invention, an embodiment that is presently preferred, is discussed in more detail with additional reference to the figures.

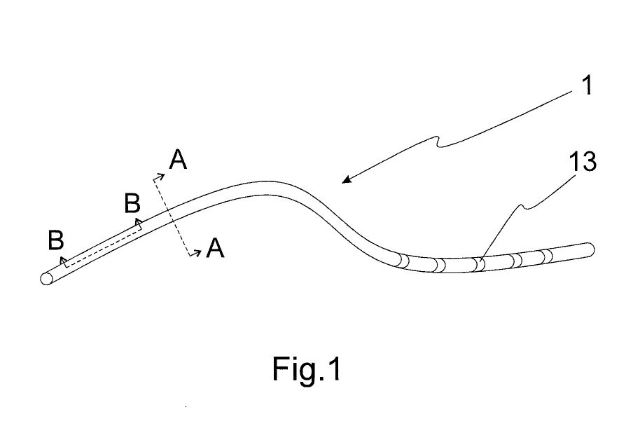

FIG. 1 shows a perspective view of an embodiment of the medical device 1 where the main body is a wire. The shown medical device 1 may be used in the Seldinger technique. The medical device 1 is shown in an arbitrary configuration where the bending around the middle part of the medical device 1 should illustrate the flexibility of the medical device 1. The medical device 1 comprises visible marks 13 arranged at certain distances to each other in the vicinity of one end of the medical device 1. When the medical device 1 is inserted into a body from the other end, the visible marks 13 enable the operator to recognize the length of the already inserted part of the medical device 1. The visible marks 13 may be arranged at a certain part of the medical device 1, as shown in the figure, or they may be arranged across the whole surface of the medical device 1.

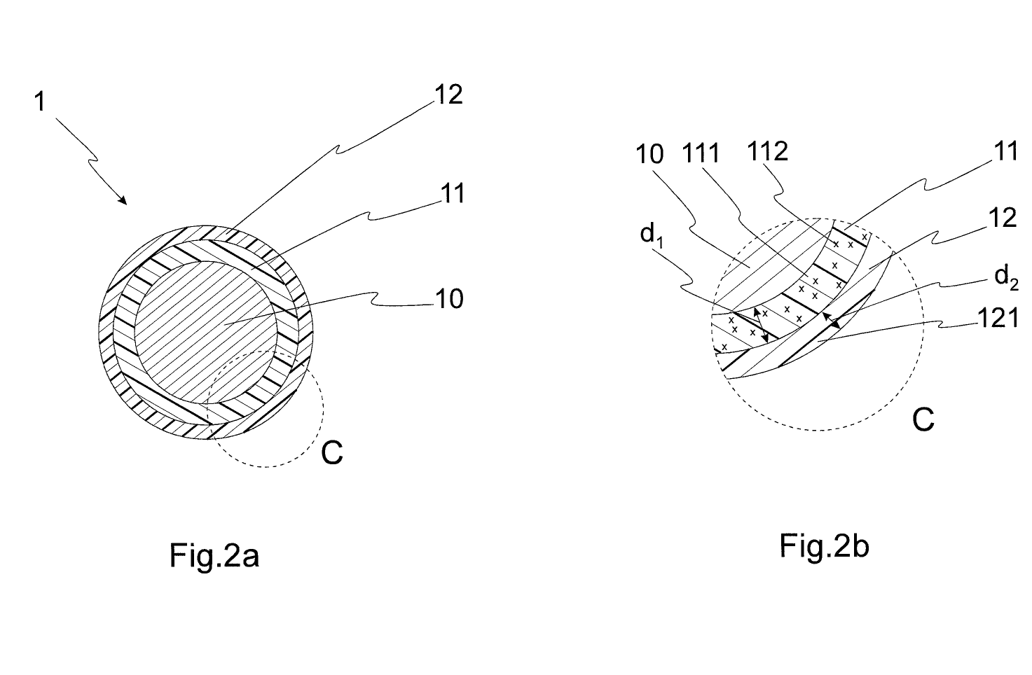

FIG. 2 shows a cut view of the medical device 1 along line A-A in FIG. 1. The medical device 1 comprises a wire 10 as a main body, a first coating 11 and a second coating 12. In the shown embodiment, the first coating 11 completely encases the wire 10 in radial direction. The first coating 11 in turn is completely encased by the second coating 12. The shown diameters of the wire 10 and the thickness of the coatings 11 and 12 are only schematically and do not reflect the real ratios of the diameter and the thicknesses.

FIG. 2b shows a magnification view of the circle marked by C in FIG. 2a. The first coating 11 comprises a matrix 111 which may be PTFE. The matrix 111 is at least partially translucent. The first coating 11 further comprises luminescent particles 112 which are embedded in the matrix 111. The luminescent particles 112 may be fluorescent and/or phosphorescent particles. The first coating 11 has a thickness d₁ around 50 μm for a wire 10 which is coated by paint enamelling. The wire 10 has a diameter around 0.68 mm. The first coating 11 is only shown schematically and could consist of several layers with thicknesses around 14 μm each. The shown medical device 1 further comprises a second coating 12 which comprises a matrix 121. The matrix 121 may be clear PTFE without luminescent particles. The second coating 12 has a thickness d₂ around 20 μm.

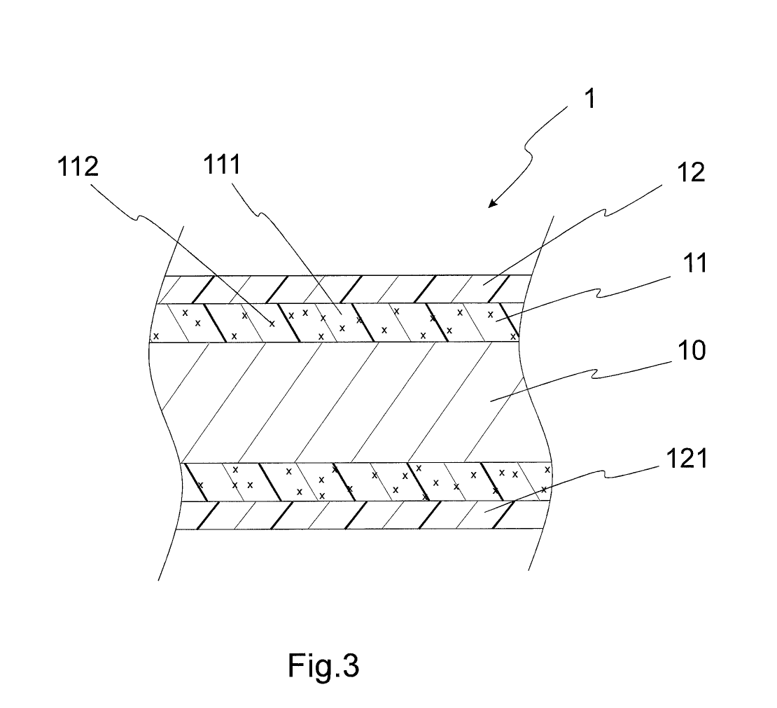

FIG. 3 shows a cut view of the medical device 1 along line B-B in FIG. 1. As seen in FIGS. 2a and 2b, the medical device 1 comprises a wire 10 as a main body, a first coating 11 with a matrix 111 and luminescent particles 112, and a second coating 121.

1. A medical device with enhanced visibility for insertion into a human or animal body and/or for contact with human or animal tissue, comprising:
a main body and at least one first coating at least partially covering the main body, the at least one first coating comprising:
an at least partially translucent matrix and;
at least one luminescent component.

2. The medical device according to claim 1, further comprising at least one second coating, the second coating:
comprising an at least partially translucent matrix; and
encasing the at least one first coating.

3. The medical device according to claim 2, wherein the second coating comprises at least one luminescent component.

4. The medical device according to claim 2, wherein the matrix of the first and/or second coating is biocompatible and is selected from the group of materials consisting of: Ethylene tetrafluoroethylene (ETFE), Polytetrafluoroethylene (PTFE), Polyesterimide, Polyimide, and combinations thereof.

5. The medical device according to claim 1, wherein the at least one luminescent component comprises fluorescent and/or phosphorescent particles.

6. The medical device according to claim 5, wherein the fluorescent and/or phosphorescent particles are of a size of between 10 microns (μm) and 30 microns (μm).

7. The medical device according to claim 2, wherein the matrix of the first and/or second coating comprises enamel varnish.

8. The medical device according to claim 7, wherein the weight ratio of the luminescent component to the enamel varnish is in a range of 0.1:1 to 2:1.

9. The medical device according to claim 2, wherein the matrix of a coating composition for the first and/or second coating comprises a thinner.

10. The medical device according to claim 1, wherein the main body is selected from the group consisting of a wire, a needle, a tube, and a rod.

11. The medical device according to claim 1, wherein the medical device is flexible.

12. The medical device according to claim 1, further comprising visible marks indicating spatial distances on the medical device.

13. A method of coating a medical device, comprising:
a. providing a main body;
b. providing a coating composition by mixing a matrix material and at least one luminescent component;
c. applying the coating composition to at least a part of the main body; and
d. finishing the coating composition.

14. The method according to claim 13, wherein the applying the coating composition is selected from the group consisting of paint enameling, dip coating, spray coating, brush coating, powder coating, and combinations thereof.

15. The method according to claim 13, wherein the finishing of the coating composition is selected from the group consisting of curing with UV light, curing with blue light, curing with air, curing with heat, and combinations thereof.

16. The method according to claim 13, further comprising a step of generating visible marks at specific positions on the medical device.

17. The method according to claim 13, further comprising providing and applying a second coating composition over the coating composition.

18. The medical device according to claim 7, wherein the enamel varnish comprises Ethylene tetrafluoroethylene (ETFE) or Polytetrafluoroethylene (PTFE).

19. The medical device according to claim 9, wherein the thinner comprises Xylene.

20. The medical device according to claim 10, wherein the main body is made of metal.

21. The method according to claim 16, wherein the generating visible marks is selected from the group consisting of laser treatment, painting, spraying, burning, and combinations thereof.
